(19) United States
(12) Patent Application Publication
(10) Pub. No.: US 2025/0064770 A1
(43) Pub. Date: Feb. 27, 2025

(54) METHOD FOR PREVENTING OR TREATING COCAINE ADDICTION AND APPLICATIONS THEREOF

(71) Applicant: PHARWAR BIOTECHNOLOGY, INC. PTE. LTD., SINGAPORE (SG)

(72) Inventor: KOU-JUEY WU, Taoyuan City (TW)

(21) Appl. No.: 18/946,263

(22) Filed: Nov. 13, 2024

Related U.S. Application Data
(63) Continuation-in-part of application No. PCT/CN2023/088337, filed on Apr. 14, 2023.
Application No. PCT/CN2023/088337 is a continuation of application No. 63/358,954, filed on Jul. 7, 2022.

(51) Int. Cl. A61K 31/222; A61P 25/30

(52) U.S. Cl. CPC A61K 31/222; A61P 25/30

(57) ABSTRACT

Provided are methods for preventing or treating cocaine addiction, reducing the likelihood of a relapse of cocaine addiction, ameliorating a physical symptom or defect of cocaine addiction, and neuroregeneration. The above methods comprise administering an effective amount of a SIAH1 inhibitor to a subject in need thereof.

### CROSS-REFERENCE TO RELATED APPLICATIONS

This application is a continuation-in-part (CIP) application of International Application No. PCT/CN2023/088337 filed on Apr. 14, 2023, which claims priority from U.S. Provisional Patent Application No. 63/358,954, filed on Jul. 7, 2022, and the entire content of which is incorporated by reference herein.

### BACKGROUND OF THE INVENTION

### 1. Field of the Invention

The present invention relates to a method for preventing or treating cocaine addiction.

### 2. Description of the Prior Arts

Cocaine, a compound extracted from the leaves of the coca plant (Erythroxylum coca) native to South America, is the second most used narcotics in the world. It is most commonly used in the United States, Europe and South America. Cocaine can produce extremely pleasurable effects on users, bring them happiness, excitement and confidence, all of which make cocaine a highly addictive drug.

However, cocaine also causes serious side effects on users. Weight loss, change of eating behavior, and anxiety are considered to be related to cocaine use. Moreover, cocaine's major complications comprise hypertension, heart rate increase and the like, which may lead to sudden death. Number of deaths caused by cocaine abuse in the United States in 2020 is 19,447.

There is no effective corresponding therapeutic medicament for cocaine addiction. Therefore, it is necessary to develop a method for effectively treating or ameliorating cocaine addiction.

### SUMMARY OF THE INVENTION

According to the aforementioned shortcomings, one objective of the present invention is to provide a method for treating or preventing cocaine addiction.

To achieve the aforementioned objective, the present invention provides a method for preventing or treating cocaine addiction, comprising administering an effective amount of a SIAH1 inhibitor to a subject in need thereof.

The present application further provides a SIAH1 inhibitor for use in preventing or treating cocaine addiction.

According to the present invention, the SIAH1 inhibitor is Sivelestat (SIV), or a pharmaceutically acceptable salt or solvate thereof. The CAS no. of Sivelestat is 127373-66-4. The acceptable salts could be in chloride, bromide, sulfate, phosphate, or acetate form.

According to the present invention, the subject is a human.

According to the present invention, the term “effective amount” refers to a dosage which effectively achieves desired treatment, prevention or amelioration of cocaine addiction during a required period of time. According to the present invention, it refers to administering a specific amount range of the SIAH1 inhibitor, such as SIV, which can decrease the increased anxiety-like behavior, such as locomotion activity induced by cocaine, can ameliorate the cocaine addiction, such as decreasing the time spent in cocaine paired compartment in CPP test, or decreasing the lever press frequencies induced by cocaine, and can rescue the Fr structure and Fr function damaged by cocaine.

According to the present invention, the effective amount of the aforementioned compound is 0.01 mg/kg/day to 100 mg/kg/day. Preferably, the effective amount of the aforementioned compound is 0.8 mg/kg/day to 2 mg/kg/day. The above dosage is calculated in accordance with the guidance document “Estimating the Maximum Safe Starting Dose in Initial Clinical Trials for Therapeutics in Adult Healthy Volunteers” published by the U.S. Food and Drug Administration in 2005.

According to the present invention, the effective amount of the aforementioned compound is 0.1 mg/kg/day to 50 mg/kg/day, for example, 0.5 mg/kg/day to 20 mg/kg/day or 0.7 mg/kg/day to 10 mg/kg/day.

The present application further provides a method for modulating the enzymatic activity of SIAH1, comprising administering to a subject in need thereof an effective amount of SIV or a pharmaceutically acceptable salt or solvate thereof.

The present invention further provides a method for preventing a relapse of cocaine addiction, comprising administering an effective amount of a SIAH1 inhibitor to a subject in need thereof.

The present invention further provides a SIAH1 inhibitor for use in preventing a relapse of cocaine addiction.

The present invention further provides a method for neuroregeneration, comprising administering an effective amount of a SIAH1 inhibitor to a subject in need thereof.

The present invention further provides a SIAH1 inhibitor for use in neuroregeneration.

According to the present invention, the neuroregeneration includes regeneration of Fr.

The present invention further provides a method for ameliorating a physical symptom or defect of cocaine addiction, comprising administering an effective amount of a SIAH1 inhibitor to a subject in need thereof.

The present invention further provides a SIAH1 inhibitor for use in ameliorating a physical symptom or defect of cocaine addiction.

According to the present invention, the physical symptom or defect of cocaine addiction includes increased heart rate, increased blood pressure, lesion of Fr, or a combination thereof. The lesion of Fr comprises function damage and structure damage.

According to the present invention, a SIAH1 inhibitor, especially SIV, can treat, prevent or ameliorate the cocaine addiction.

### BRIEF DESCRIPTION OF THE DRAWINGS

FIG. 1A shows body weight of wild-type (WT) and Hausp^{K444R} point mutant mouse strain;

FIG. 1B shows the food intake of wild-type (WT) and Hausp^{K444R} point mutant mouse strains over time;

FIGS. 2A, 2B and 2C show the results of open field tests of the Hausp^{K444R} mice and the wild-type (WT) mice without habitation;

FIGS. 2D and 2E show the results of open field tests of the Hausp^{K444R} mice and the wild-type (WT) mice with habitation;

FIGS. 3A to 3C show the comparison between the Hausp^{K444R} mice and the wild-type (WT) mice in the dark/light box transition test;

FIGS. 3D and 3E show the comparison between the Hausp^{K444R} mice and the wild-type (WT) mice in the forced swim test;

FIG. 4A shows the dopamine levels in the Hausp^{K444R} mice brains and the wild-type (WT) mice;

FIG. 4B is the western blot analysis result of the phosphorylation levels of tyrosine hydroxylase (TH, p-TH) and its downstream targets (CDK5, p35/25) in the Hausp^{K444R} mice brain tissue and in the wild-type (WT) mice brain tissue;

FIG. 4C is the SIK2 level in the Hausp^{K444R} mice brain tissue and in the wild-type (WT) mice brain tissue;

FIG. 4D shows the HAUSP level, SIK2 level, p-TH level and p35/25 level in HAUSP knockdown human SH-SY5Y cells (HAUSP-si) and in control (scr-si) SH-SY5Y cells;

FIG. 4E shows the HAUSP level, SIK2 level, p-TH level and p35/25 level in HAUSP overexpressed human SH-SY5Y cells (OE-HAUSP) and in control (vec-ctrl) SH-SY5Y cells;

FIG. 5A shows the levels of PNMT in the Hausp^{K444R} mice brain tissue and in the wild-type mice brain tissue;

FIG. 5B shows the levels of ΔFosB in the Hausp^{K444R} mice brain tissue and in the wild-type (Hausp^{WT}) mice brain tissue;

FIG. 5C shows the ΔFosB levels in HAUSP overexpressed human SH-SY5Y cells (OE-HAUSP) and in control (vec-Ctrl.) SH-SY5Y cells;

FIG. 6A shows the co-immunoprecipitation experiment results of the interaction between HAUSP and SIK2 using SH-SY5Y cells;

FIG. 6B is the GST pull down assay results of the interaction between HAUSP and SIK2;

FIG. 6C is the in vitro deubiquitination assay result using wild-type HAUSP and mutant HAUSP to deubiquitinate the polyubiquitinated Flag-SIK2 in SH-SY5Y cells;

FIG. 6D shows the maintenance of SIK2 levels by wild-type HAUSP, but not by mutant HAUSP, in SH-SY5Y cells under cycloheximide (CHX) treatment;

FIG. 7A shows the Fasciculus retroflexus (Fr) structure in the Hausp^{K444R} mice vs. the wild-type mice by H&E and LFB staining and their magnifications are shown as below;

FIG. 7B shows the Fr structure in the Hausp^{K444R} mice vs. the wild-type mice by silver staining, wherein MHb is Medial Habenula; Sm is stria medullaris; LHb is Lateral Habenula; and HP is Hippocampus;

FIG. 7C is the 3-Amino-9-Ethylcarbazole (AEC) staining of Fr structure in the E14 brain of Hausp^{K444R} mice vs. the wild-type mice;

FIG. 8A is the staining of Fr structure by staining with either anti-Neuropilin-2 antibody or anti-ROBO-1 antibody in the E14 and 4-week old mice brain tissue;

FIG. 8B is the staining of Fr structure with anti-Neuropilin-2 antibodies in 4-week old mice brain tissue;

FIG. 9A is the Masson's trichrome staining of Fr structure with Masson's trichrome in the Hausp^{K444R} mice brain tissue vs. the wild-type mice brain tissue and their magnifications are shown as below;

FIG. 9B is the Masson's trichrome staining of Mtt (Mammillothalamic tract) structure with Masson's trichrome in the Hausp^{K444R} mice brain tissue vs. the wild-type mice brain tissue and their magnifications are shown as below;

FIG. 9C is the staining of Fr structure with anti-Nefm (Neurofilament medium chain) antibody in the 4-week old mice brain tissue;

FIG. 10A is an optical cleared brain image of mice whole brain staining with NRP2 (neuropilin-2)/TH (tyrosine hydroxylase) followed by imaging;

FIG. 10B is an optical cleared brain image of mice whole brain staining with NRP2 (neuropilin-2) followed by imaging;

FIG. 10C shows 3D visualization images of the Fr structure in wild-type mice or in the wild-type mice treated with cocaine (images shown for each mouse are the same structures looking from different angles);

FIG. 10D shows the difference of Fr diameter in wild-type mice treated with cocaine or the Hausp^{K444R} mice compared to the control wild-type mice, wherein Hausp^{WT}: N=7, Hausp^{WT} treated with cocaine: N=7, Hausp^{K444R}: N=15;

FIG. 10E shows the difference of Fr length in wild-type mice treated with cocaine or the Hausp^{K444R} mice compared to the control wild-type mice, wherein Hausp^{WT}: N=12, Hausp^{WT} treated with cocaine: N=10, Hausp^{K444R}: N=20;

FIG. 11A shows the relationship between cocaine treatment and the levels of Hausp and downstream proteins in mouse brain tissues from three different mice (triplicates);

FIG. 11B is the co-immunoprecipitation assay result showing the interaction between HAUSP and SIAH1;

FIG. 11C shows the influence on HAUSP levels by overexpression of SIAH1;

FIG. 12A shows the influence of SIV on the relationship between cocaine treatment and the levels of Hausp and downstream proteins in human SH-SY5Y cells;

FIG. 12B is the NrCAM immunohistochemistry of the influence of SIV treatment on the Fr structure in the Hausp^{K444R} mice brain compared to the Hausp^{K444R} mice brain without SIV treatment;

FIGS. 13A, 13B, and 13C show the efficacy of SIV (100 mg/kg) to treat mice induced by cocaine in locomotion activity test;

FIG. 13D shows the efficacy of SIV (100 mg/kg) to treat mice induced by cocaine in CPP test;

FIG. 14A shows the number of lever press induced by cocaine after administration of SIV (100 mg/kg) vs. administration of vehicle as a control; numbers 1 to 6 represent different mice, respectively;

FIG. 14B is the bar graph showing that the treatment of SIV (100 mg/kg) reduces lever responses induced by cocaine compared to vehicle group;

FIG. 14C is the efficacy of SIV (100 mg/kg) for cumulative cocaine reinforcements;

FIG. 14D is the latency to first response in mice induced by cocaine after administration of SIV (100 mg/kg) compared to vehicle group;

FIG. 15A shows the efficacy of SIV (50 μg/ml or 100 μg/ml) to inhibit the K48-linked polyubiquitination of HAUSP mediated by SIAH1 in E3 ligase auto-ubiquitination assay compared to vehicle group; and

FIG. 15B shows the western blot results which demonstrate the efficacy of SIV (50 μg/ml or 100 μg/ml) to restore the HAUSP levels decreased by cocaine in SH-SY5Y cells.

### DETAILED DESCRIPTION OF THE PREFERRED EMBODIMENTS

The present invention is further explained through the following embodiments. The present invention should not be limited to the contents of the embodiments. A person having ordinary skill in the art can make some improvement or modifications which are not departing from the scope of the present invention.

### Preparation Example 1: Cocaine Addiction Animal Model-Hausp^{K444R} Point Mutation Mouse

A mouse model is generated with Hausp^{K444R} point mutation in mouse embryonic stem cells (ES cell) in accordance with the method described by Wu, Han-Tsang, et al. (K63-polyubiquitinated HAUSP deubiquitinates HIF-1α and dictates H3K56 acetylation promoting hypoxia-induced tumour progression. Nature communications, 7(1), 1-17 (2016): a homozygous Hausp K444R point mutant (corresponding to K443R mutation in HAUSP) mouse strain by a conventional knock-in approach was provided. The insertion of the L1L2_Bact_P cassette created a knock-in mutant of exon 13, at position 444, Lysine (K) to Arginine (R), of Hausp in Chromosome 16 (GRCm39, C57BL/6J). The cassette was composed of an FRT site followed by lacZ sequence and a loxP site. This first loxP site was followed by a neomycin resistance gene under the control of the mouse beta-actin promoter, SV40 polyA, a second FRT site and a second loxP site. Cre deletes the promoter-driven selection cassette and floxed exon of the tm1 allele to generate a lacZ-tagged allele. It should be noted that: (a) the point mutation strategy was used to generate Hausp K444R point mutant in mouse ES cells; and (b) the Hausp K444R mutation (A to G) in mouse skin fibroblast cells was identified by sequencing and PCR analysis. cDNA products from wild-type (Hausp^{+/+}) and from K443R homozygous (HAUSP^{K443R/K443R}) were amplified by PCR using HAUSP typing primers to generate the 594 bp (WT) and 694 bp (K444R) products. The mouse model was used in the following animal experiments.

### Preparation Example 2: Knockdown of HAUSP in Human SH-SY5Y Cells

Lentivirus containing short hairpin RNAs (shRNAs) expressed in a lentiviral vector (pLKO.1-puro) were generated in 293T cells. For lentivirus production, 293T cells were transfected with 15 mg pLKO.1-puro lentiviral vectors expressing different shRNAs along with 1.5 mg of envelope plasmid pMD.G and 15 mg of packaging plasmid pCMVDR8.91. Virus was collected 48 h and 72 h after transfection. The sequence and clonal names of plasmid were pLKO-scrambled or another pLKO plasmid against HAUSP (shRNA HAUSP-si 1: RNAi core, sinica, Taiwan-TRCN0000004059; shRNA HAUSP-si 2: RNAi core, sinica, Taiwan-TRCN0000010845). To prepare the HAUSP knockdown cell, SH-SY5Y cells were infected with lentivirus for 24 h, and stable clones were generated and selected by appropriate antibiotics.

### Preparation Example 3: Overexpression of HAUSP in Human SH-SY5Y Cells

The HAUSP plasmids were obtained from Prof. Wei Gu (Columbia University) (Wu, Han-Tsang, et al. (K63-polyubiquitinated HAUSP deubiquitinates HIF-1α and dictates H3K56 acetylation promoting hypoxia-induced tumour progression. Nature communications, 7(1), 1-17 (2016). Lipofectamine method was used to transfect plasmids. Briefly, plasmids were first incubated with Lipofectamine reagent within 20 mins and further incubated with human SH-SY5Y cells for 48 hrs.

### Experiment 1: Body Weight and Food Intake of Experiment Animal

25 three-week-old Hausp^{K444R} point mutant mice from preparation Example 1 and 28 wild-type C57BL/6j mice were housed under conditions of controlled humidity (55±10%) and temperature (22±2° C.) with ad libitum feeding after starving on Day 0. Day 0 is the day that the mice were fasted for 14 hours (hr) to 18 hr (overnight fasting). The body weights of mice were measured before and during the experiment. Moreover, the food intake by the mice was recorded as well.

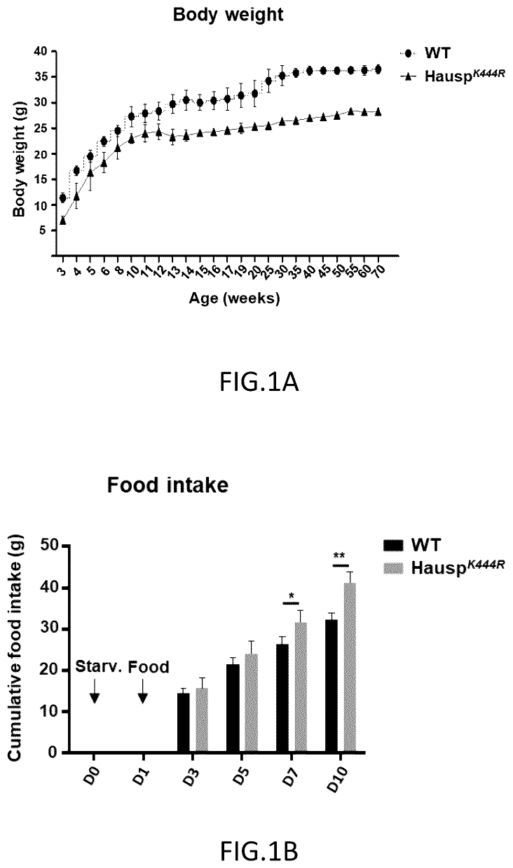

The results can be seen from FIG. 1A to FIG. 1B, which show that the Hausp^{K444R} mice had less body weight compared to the wild-type (WT) mice (FIG. 1A) and food intake of Hausp^{K444R} mice were more than that of the wild-type mice (FIG. 1B).

### Experiment 2: Locomotion Activity I: Open Field Test

8 mice from Preparation Example 1 were randomly chosen to conduct Locomotion Activity I. The open fields using white acrylic chambers measured 50 cm (length)×50 cm (width)×38 cm (height) were made from white high density and non-porous plastics. Once the Video Tracking software (ANY-maze, Stoelting Co, IL, USA) was opened, the cursor was moved to the “Single-Subject Tracking” option located under the “Data Acquisition” tab and single-clicked to open this option. The software package used in this protocol allows the tracking of up to 6 individual mice at one time. The mice were brought to open fields to acclimate to the procedure room for 30 minutes (min) prior to starting the test. The tracking plot of randomly chosen 6 mice was recorded and analyzed by ANY-maze software.

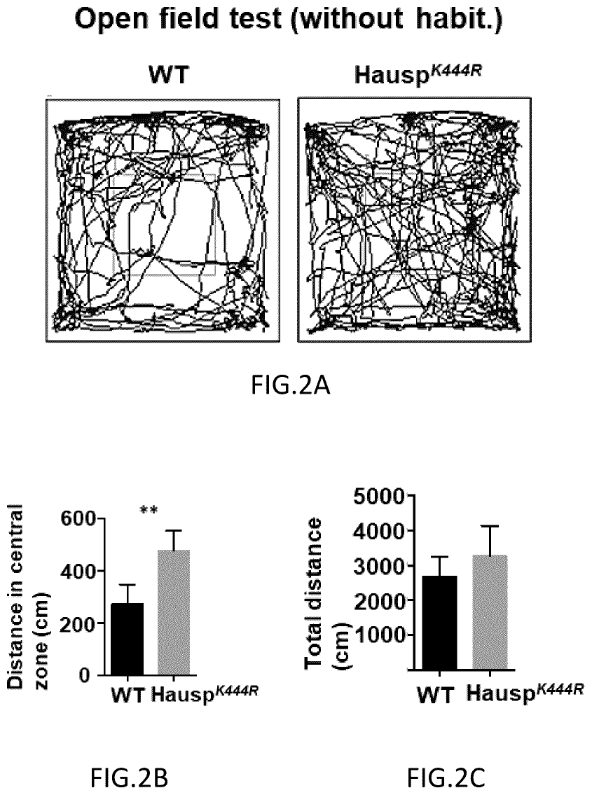

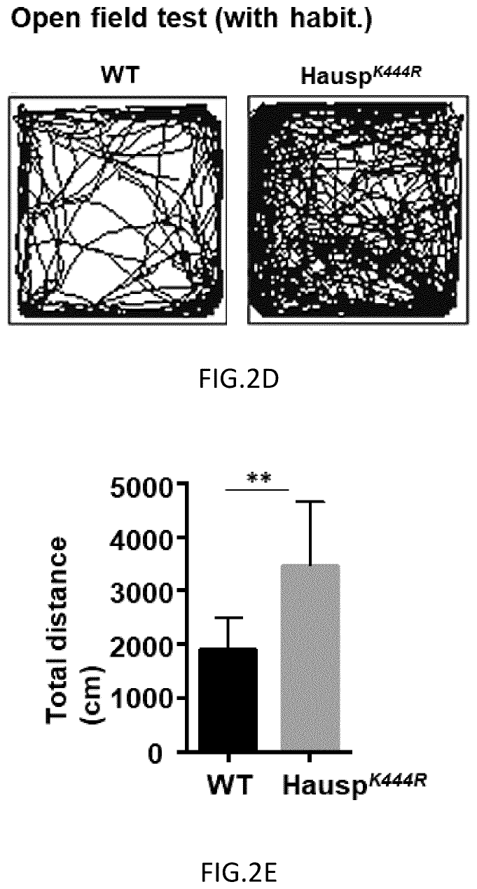

FIG. 2A to FIG. 2C show that the Hausp^{K444R} mice without habituation had higher activity than the wild-type (WT) mice. It can also be seen from FIG. 2A to FIG. 2C that the Hausp^{K444R} mice (without habituation) ran across the central zone of the space more often and had much higher total movement distance than the wild-type mice. That is, the Hausp^{K444R} mice are similar to cocaine addiction mice due to their high activity and behavior of staying more time in the central zone of space, which is not the same with the normal mice. The results of similar experiments (when mice were put with habituation) were shown in FIG. 2D and FIG. 2E.

### Experiment 3: Locomotion Activity II: Light-Dark Transition Test and Forced Swim Test

The light-dark test is a widely used behavioral test to assess anxiety-like behavior in rodents, including mice. This test is based on the natural aversion of rodents to brightly lit, open spaces and their preference for dark, enclosed spaces. The test apparatus consists of two compartments, one light and one dark, which are connected by an opening. During the test, a mouse was placed in the dark compartment and allowed to explore the apparatus freely for 10 mins. The amount of time (in second (s)) the mouse spent in the light compartment (anxiety-reducing behavior) versus the dark compartment (anxiety-increasing behavior) and the number of entries were recorded and measured. 6 mice from Preparation Example 1 were randomly chosen to conduct light-dark Transition Test. The wild-type group contained 6 C57BL/6j mice.

The forced swim test involved placing a mouse in a container filled with water and observing its behavior for 5 mins. The latency of first immobility and total immobility were recorded in a unit of seconds (s). 6 mice from Preparation Example 1 were randomly chosen to conduct forced swim Test. The wild-type group contained 6 C57BL/6j mice.

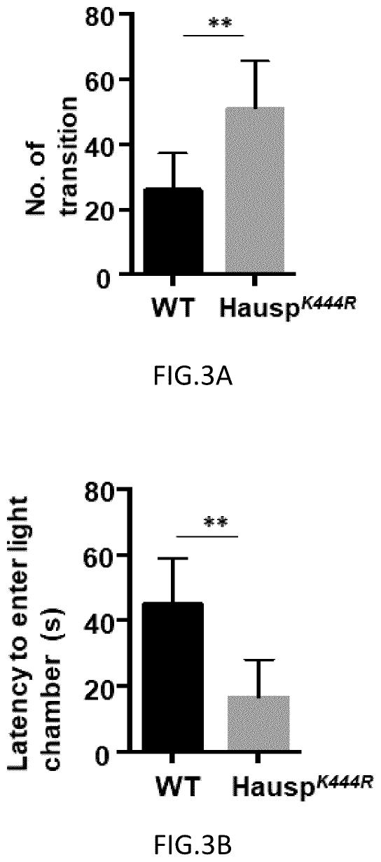

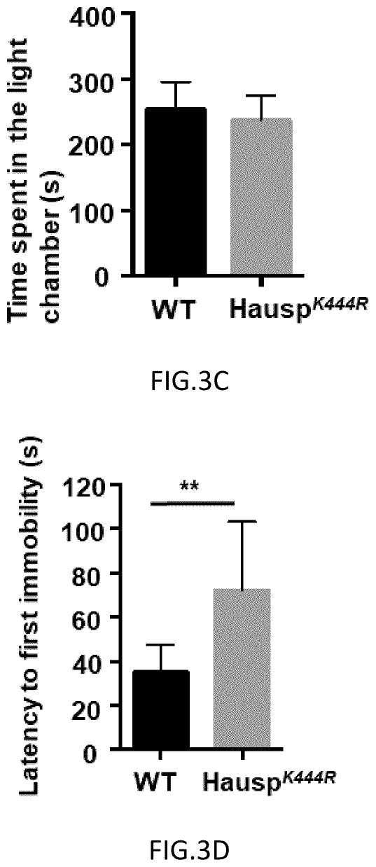

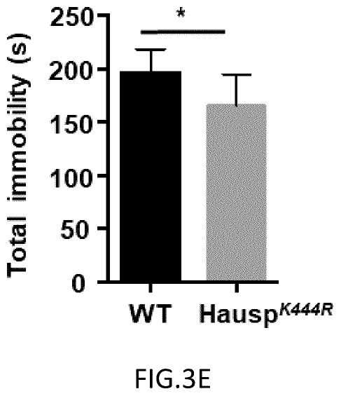

The results of light-dark test and the forced swim test are respectively shown in FIGS. 3A to 3E, and also shown in the following Table 2.

It can be seen from FIGS. 3A to 3C, that the Hausp^{K444R} mice had higher number of transitions and shorter latency to enter light chamber than that of the wild-type mice. This indicates that the Hausp^{K444R} mice were more anxious than the wild-type mice and exhibiting the symptom of anxiety of cocaine addiction.

FIG. 3D and FIG. 3E show that the Hausp^{K444R} mice had longer latency to first immobility and shorter total immobility than the wild type mice in forced swim test.

TABLE 2
Time period of first immobility and the time period of total
immobility of the Hausp^{K444R} mice and wild type mice

| | Latency to first immobility (s) | Total immobility (s) |
|---|---|---|
| Wild-type | 35.13 ± 1.377 | 196.3 ± 7.873 |
| Hausp^{K444R} | 72.30 ± 9.781** | 165.0 ± 9.396* |

The results of the Table 2 show that the time period of first immobility were longer in the Hausp^{K444R} mice than those of the wild-type mice and the total immobility of the Hausp^{K444R} mice was shorter than those of the wild-type mice. This indicates that the Hausp^{K444R} mice were more active than wild-type mice and exhibiting the symptom of cocaine addiction.

The results of Experiments 1 to 3 prove that the Hausp^{K444R} mice have the behavior similar to cocaine addiction mice.

Experiment 4: Mouse Microdialysis Test, Protein Extraction and Western Blot Assay: TH, CDK5, p35/25, SIK2 and HAUSP, and Immunohistochemistry

The mouse microdialysis test is a technique used in neuroscience research to measure the levels of neurotransmitters, hormones, and other molecules in the brain. The microdialysis probe is a thin, flexible tube inserted into the nucleus accumbens in the brain, which allows for the collection of small amount of extracellular fluid (ECF) from the surrounding tissue. Dopamine is analyzed and measured in the ECF samples from Hausp^{K444R} mouse brains or the wild-type group. The Application of On-line Microdialysis with High Performance Liquid Chromatography (DECADE™ Elite, Netherland) was used for determining dopamine in the Hausp^{K444R} mouse brains compared to wild-type group.

As for protein extraction and western blot assay, lysis buffer (T-PER™ and M-PER™, Thermo Fisher Scientific Inc., USA) containing protease inhibitors was used for extraction of proteins: tyrosine hydroxylase (TH), Cyclin-dependent kinase 5 (CDK5), p35/25, Salt Inducible Kinase 2 (SIK2) from mouse brain tissue and cell lines of SH-SY5Y. Lysates were clarified by centrifugation at 13,000 rpm, 4° C. for 10 min. The protein content was determined by Bradford method (Bio-Rad Laboratories, Hercules, CA, USA). For Western blot analysis, 15 μg to 25 μg protein extracts from each sample were loaded to 10% sodium dodecyl sulfate (SDS)-PAGE gels and transferred to nitrocellulose membranes. The membranes were probed with different antibodies, and an anti-β-actin or α-tubulin antibody was selected as a loading control. Signals were developed using an ECL chemiluminescence kit (Tools, USA). Molecular weight markers were labelled on the left side of each lane. Among the proteins being analyzed, Cdk5 is the protein which phosphorylates TH to regulate dopamine level. P35 is a subunit of Cdk5, which is important for the activity of Cdk5. P35 can be cleaved to P25, a protein which prolongs Cdk5 activation. SIK2 phosphorylates p35 and triggers degradation of p35. SIK2 provides a link between p35 and CDK5. The western blot assay was used for determining the protein expression in the Hausp^{K444R} mouse brains compared to wild-type group, or the protein expression of knockdown of HAUSP or overexpression of HAUSP in human SH-SY5Y cells compared to control human SH-SY5Y cells.

For immunohistochemistry, the whole brain of the Hausp^{K444R} mice and wild-type mice was equally divided into five parts, embedded in the same paraffin block in sequence for coronal section, and stained to observe the expression level of the target proteins. 4 mm thick sections of all tissues were cut from the paraffin embedded specimens, as samples for IHC staining the phosphorylated TH (p-TH) (Cell Signaling, #13041, USA), CDK5 (Cell Signaling, #14145, USA) and p35/25 (Cell Signaling, #14145, USA). The samples were fixed in 4% paraformaldehyde solution for 24 hours. The endogenous peroxidase activity was eliminated with 3% hydrogen peroxide and then incubated with 1% bovine serum albumin and 5% normal goat serum for the blocking step. After reacting with a biotinylated secondary antibody for 1.5 hours, antigen-antibody reactions were visualized using streptavidin-horseradish peroxidase conjugate (DAKO LSAB kit; DAKO, Los Angeles, CA, USA), with 3-amino-9-ethylcarbazole as the chromogen. All slides were counterstained with hematoxylin. For immunofluorescence assay, the fluorescent-conjugated secondary antibodies were used, including Alexa Flour 488 (Abcam, ab150077, UK) and 594 (Abcam, ab150116, UK).

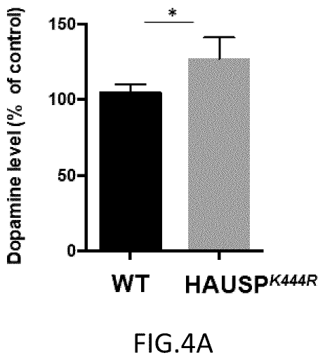

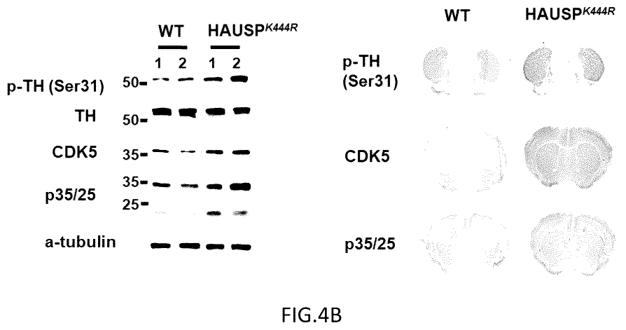

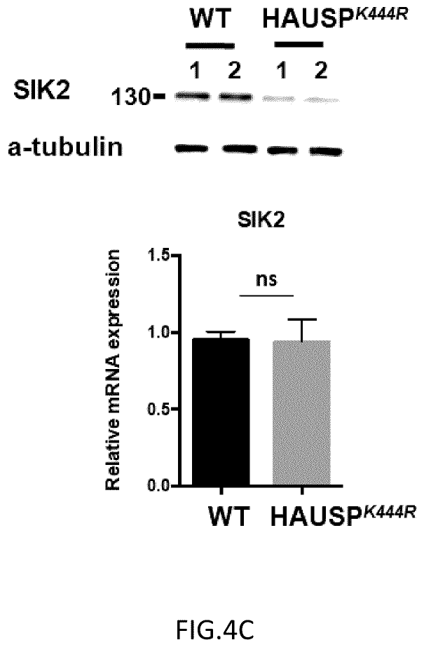

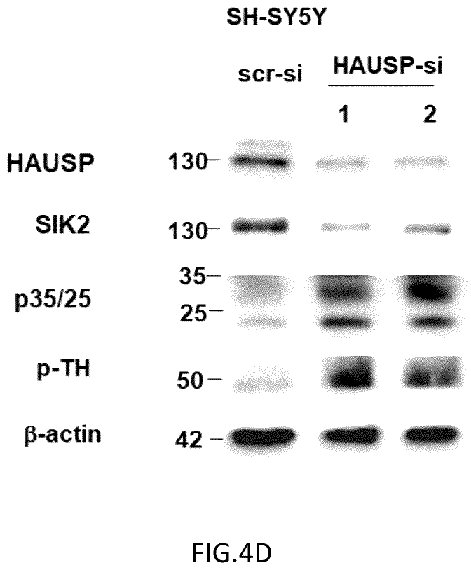

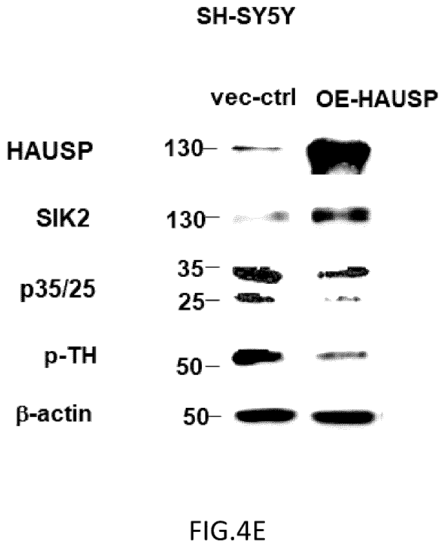

FIGS. 4A to 4E show the dopamine levels and levels of proteins involved in the signaling pathways regulated by HAUSP in the Hausp^{K444R} mice and WT mice. It can be seen from FIG. 4A that the dopamine levels in the Hausp^{K444R} mice brains were higher than those in the wild-type brain tissue.

In FIG. 4B, the result of western blot analysis and IHC staining showed that the phosphorylation levels of tyrosine hydroxylase (TH) and its downstream targets (CDK5, p35/25) were higher in the Hausp^{K444R} mice brain tissue than in the wild-type mice brain tissue.

In FIG. 4C, it is found that the SIK2 levels were lower in the Hausp^{K444R} mice brain tissue than in the wild-type mice brain tissue. It can be deduced that SIK2 might play an important role in neurotransmitter pathway of the Hausp^{K444R} mice.

Moreover, knockdown of HAUSP by two different shRNA-plasmid (HAUSP-si 1 and HAUSP-si 2) targeting different sequences of HAUSP in human SH-SY5Y cells showed a decreased SIK2 level and increased p-TH and p35/25 levels in FIG. 4D compared to control (scr-si) SH-SY5Y cells, which is consistent with the mice results. It can also be seen from the results of FIG. 4D that the HAUSP silence by two different shRNA-plasmids leads to the same downstream reaction.

On the other hand, it can be seen from FIG. 4E that overexpression of HAUSP in human SH-SY5Y cells show the upregulation of SIK2 levels and repression of p-TH and p35/25 levels compared to control (vec-ctrl) SH-SY5Y cells. These prove again that the sik2 is an important gene in neurotransmitter pathway of cocaine addiction.

### Experiment 5: Protein Extraction and Western Blot Assay: PNMT and ΔFosB, and Immunohistochemistry

The methods of protein extraction and western blot assay immunohistochemistry are similar to Example 4, and the difference lies in that the proteins to be analyzed in Experiment 5 are PNMT and ΔFosB instead.

The high motility of Hausp^{K444R} mice or cocaine addiction mice is related to high epinephrine. Epinephrine can be converted from norepinephrine by Phenylethanolamine N-methyltransferase (PNMT) (H. A. Khan, et al., 2016). Besides, it is reported that the accumulation of ΔFosB, a member of the Finkel-Biskis-Jinkins osteosarcoma (Fos) family of transcription factors encoded by the fosB gene, is accumulated in the nucleus accumbens and dorsal striatum of cocaine addiction mice.

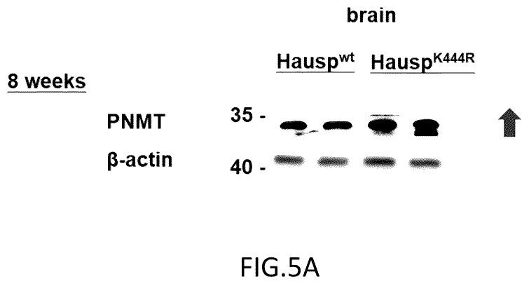

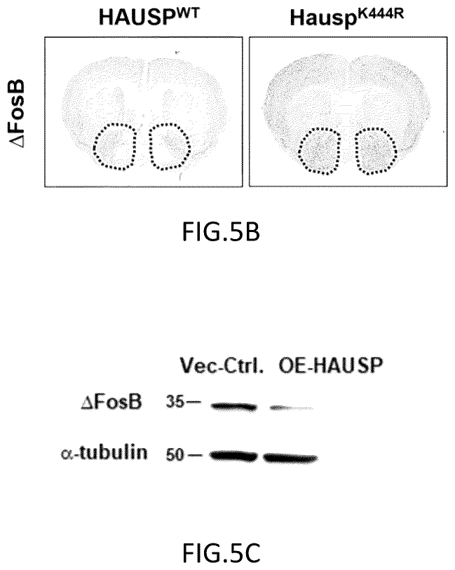

The results are shown in FIG. 5A to FIG. 5C.

It can be seen from FIG. 5A that the levels of PNMT were higher in the Hausp^{K444R} mice brain tissue than in the wild-type mice brain tissue. Therefore, it can be deduced that more epinephrine was generated in the Hausp^{K444R} mice brain, which results in high motility of the Hausp^{K444R} mice.

In FIG. 5B, the levels of ΔFosB were higher in the Hausp^{K444R} mice brain tissue than in the wild-type mice brain tissue. Therefore, it also proved that the Hausp^{K444R} mice can be used as a cocaine addiction animal model. Moreover, the overexpression of HAUSP in human SH-SY5Y cells decreases the ΔFosB levels, as shown in the result of FIG. 5C, which is also consistent with the mice results.

### Experiment 6: Co-Immunoprecipitation, GST Pull-Down and Deubiquitination Assays

Co-immunoprecipitation experiments were performed by incubating different antibodies for 5 hours with 0.5 mg to 1 mg of whole cell lysates prepared by lysis in 150 mM NaCl, 1% Nonidet P-40, 1% deoxycholate, 0.1% SDS, 50 mM Tris HCl (pH 7.5), 1 mM PMSF, 25 mM NaF and protease inhibitors to obtain an immune complex. SH-SY5Y overexpressing proteins of interest, HAUSP and SIK2, were further observed. The immune complex was incubated overnight with protein-A beads pre-blocked with 10% bovine serum albumin. The immunoprecipitates were washed three times with the same lysis buffer, mixed with 6×Laemmli dye, boiled for 6 min, and loaded on SDS-PAGE. After transfer, the filters were blocked with 5% BSA-containing blocking buffer, probed with primary (HAUSP, Bethyl, A300, USA.; SIK2, Cell Signaling, #6919, USA.) and secondary antibodies (Goat anti-mouse and rabbit, GeneTex, USA.) sequentially and developed. Input group is positive control showing that the cells contain the target protein. Knockdown of HAUSP in human SH-SY5Y cells and control human SH-SY5Y cells were used in the present experiment. IgG was used as a negative control in Co-immunoprecipitation experiments.

Glutathione S-transferase (GST) pull down assays were performed by incubating Flag-SIK2 protein (constructed by Flag-vector (FLAG® Tag Bacterial and Mammalian Expression Vectors, Sigma, USA.) and whole SIK2 gene) with GST-HAUSP 1-210 (constructed by GST-vector (pET-GST vector, Addgene, USA.) and HAUSP 1-210 fragment) or GST-HAUSP 210-500 protein (constructed by GST-vector and HAUSP 210-550 fragment) (or GST protein (Sigma, St Louis, MI, USA) as a control) and Glutathione-Agarose (Sigma, St Louis, MI, USA). Purification of GST proteins was described. The pulled down Flag-SIK2 protein was detected by Western blot analysis. The method of Western blot analysis is similar to Experiment 4; however, the protein to be analyzed is different.

For in vitro deubiquitination assay, Flag-HAUSP wild type and K443R mutants were overexpressed respectively in 293T cells and immunoprecipitated by anti-Flag agarose (Sigma). The mutation point of HAUSP in humans is HAUSP^{K443R} while the mutation point of HAUSP in mice is HAUSPK^{444R}. The flag-SIK2 was overexpressed in 293T cells, immunoprecipitated by anti-flag-antibodies (Invitrogen) and eluted by flag peptide (10 mg/ml) in the PBS. The commercial ubiquitination kit (Enzo Life Science, BML-UW9920) was used to perform this experiment. The reaction was incubated for 4 hr to 8 hr at 37° C. and subjected to Western blot analysis by using anti-K48 ubiquitin antibody.

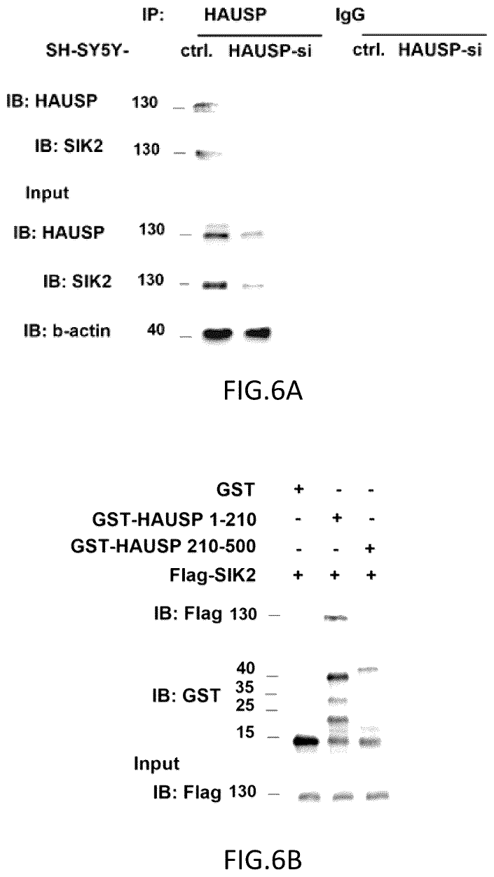

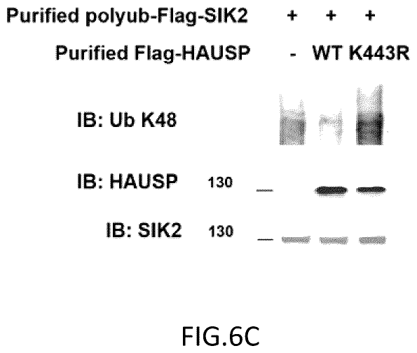

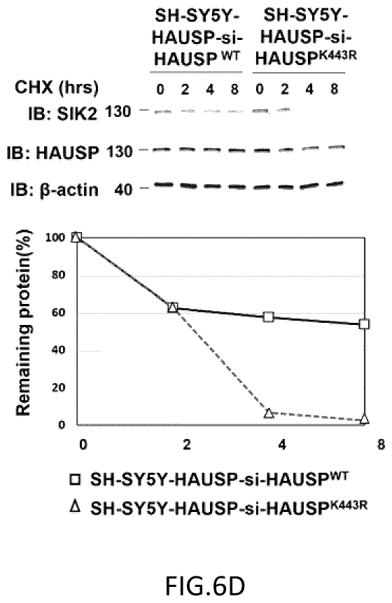

The result of the co-immunoprecipitation experiments showed the interaction between HAUSP and SIK2 using SH-SY5Y cells in FIG. 6A. The result of GST pull down assays in FIG. 6B showed the direct interaction between HAUSP and SIK2.

In FIG. 6C, wild-type HAUSP, but not the mutant HAUSP, decreased the K48-linked polyubiquitination levels of SIK2 using in vitro deubiquitination assays.

It can be seen from the results of FIG. 6D, knockdown of HAUSP in SH-SY5Y cells followed by reconstitution of wild-type HAUSP ((SH-SY5Y-HAUSP-si-HAUSP WT) delayed the degradation of SIK2 under cycloheximide treatment; whereas reconstitution with the mutant HAUSP^{K443R} (SH-SY5Y-HAUSP-si-HAUSPK⁴⁴³R) could not delay the degradation of SIK2 under cycloheximide (CHX) treatment.

### Experiment 7: Mouse Brains Analysis

Cocaine treated mice were C57BL/6j mice treated with cocaine at 10 a.m. with a dosage of 25 mg/kg/day for three months while the control mice (Hausp^{WT}) and Hausp^{K444R} mice were treated with saline. All mice were housed under conditions of controlled humidity (55±10%) and temperature (22±2° C.), with a reversed light cycle in this experiment.

After the above treatments, all organs were obtained after mouse heart perfusion by saline, and fixed by 4% paraformaldehyde solution for 24 h for H&E, LFB, Silver, Masson's trichrome staining and immunohistochemistry (IHC). Fresh tissues were immediately homogenized in T-PER™ by tissue homogenizer (HT mini homogenizer, OPS Diagnostics LLC, USA) for Western blot analysis. Handling of the animals has been approved by the Internal Animal Usage Committee of the Chang Gung Memorial Hospital at Linkou.

Mouse brains were processed following SHIELD processing protocol according to Park, Y. G. et al. 2019. Mice were perfused with ice-cold PBS and then with the SHIELD perfusion solution (ice-cold 1×PBS containing 4% (w/v) PFA and GE38 (polyglycerol 3-polyglycidyl ether) supernatant). Collected brains were incubated in the same perfusion solution at 4° C. for two days. Tissues were subsequently transferred to the SHIELD-OFF solution (ice-cold 1×PBS containing GE38 supernatant) and incubated at 4° C. for one day, followed by incubation in SHIELD-ON solution (0.1 M sodium carbonate buffer at pH 10, prewarmed to 37° C.) at 37° C. for one day. All the reagents were prepared from SHIELD kits (LifeCanvas Technologies, Seoul, South Korea) according to the manufacturer's instruction. The SHIELD-processed brains were cleared using stochastic electro-transport (SmartClear Pro II, LifeCanvas Technologies, Seoul, South Korea) with a constant current 1.5 A for 3-5 days. The cleared brains were washed thoroughly in PBS at room temperature for at least overnight and immune-stained using an adapted version of eFLASH method according to Yun, D. H. et al. 2019. Brains were pre-incubated in sample buffer at room temperature overnight. For immuno-staining, the cleared brains were placed in SmartLabel System (LifeCanvas Technologies, Seoul, South Korea) with 9 ml sample buffer in a large sample cup for each sample. For dopaminergic neurons and habenula labeling, mouse anti-TH antibody (Cell Signaling, #45648, USA) and rabbit anti-NRP2 antibody (Cell Signaling, #3366, USA) were used for each whole brain. Immuno-labeled brains were washed in PBS with Tween 20 (PBST) for 6 hours, post-fixed, and counterstained with paraformaldehyde (PFA) solution at room temperature for two days, and then washed in PBST to remove residual PFA. For Refractive Index (RI)-matching, brains were immersed in 50% CUBIC-R+ solution (Tainaka, K. et al. 2018) for one day and then in 100% CUBIC-R+ solution for another day. The optically cleared brains were imaged with a light-sheet microscope (SmartSPIM, LifeCanvas Technologies, Seoul, South Korea) using 4× objectives. For 3D visualization and habenula segmentation, Imaris software (Bitplane, Belfast, U.K.) was employed. Images were reconstructed to a multi-channel-z stack using Imaris file converter, and the regions of the whole brain, habenula region were masked and quantified using the surface function in Imaris software version 9.5. It has been reported that the cocaine treatment causes degeneration of the Fr. The outer region of Fr originates in the lateral habenula, which plays a critical role in the brain's response to reward.

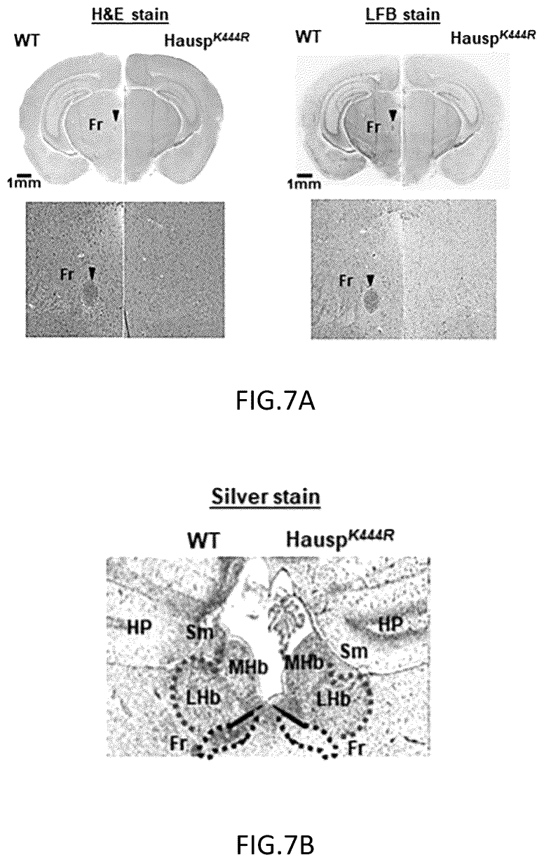

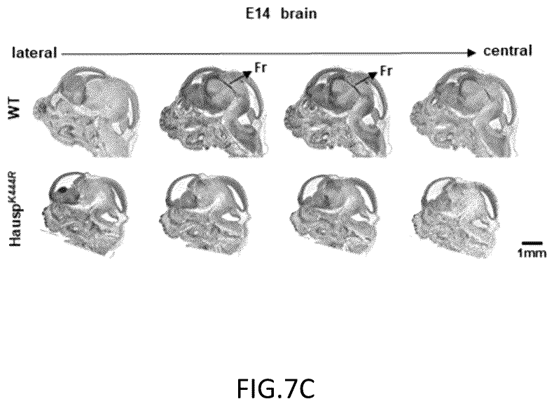

The result of FIG. 7A shows that the decreased staining of Fr in the Hausp^{K444R} mice compared to the staining of the wild-type mice by H&E and LFB staining, and the result of FIG. 7B shows the decreased staining of Fr in the Hausp^{K444R} mice compared to the wild-type mice by silver staining. Moreover, it can be found that the staining of Fr decreased in the embryonic day 14 (E14) brain of Hausp^{K444R} mice compared to the wild-type mice in FIG. 7C. According to the above results, the Fr lesion in the Hausp^{K444R} mice can be observed. This again proves that the Hausp^{K444R} mice can be used as a cocaine addiction model.

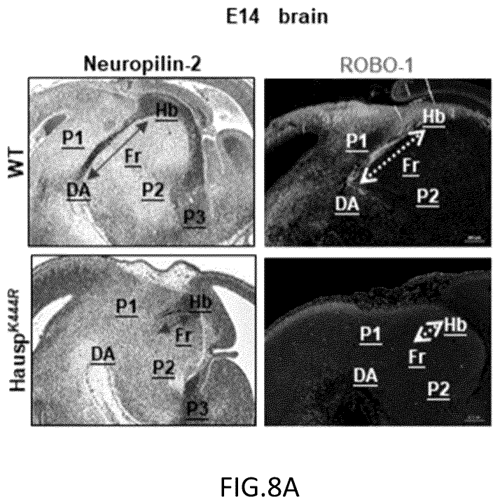

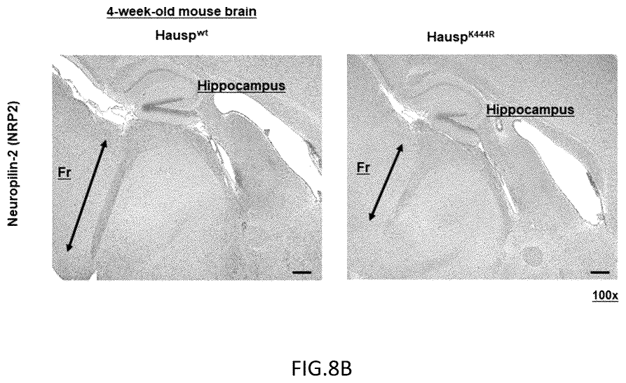

The decreased staining of Fr structure by staining with different markers in the E14 and 4-week old mice brain tissue can be found in the results of FIG. 8A and FIG. 8B. Staining with either anti-Neuropilin-2 antibody or anti-ROBO-1 antibody in FIG. 8A showed that the Fr structure was diminished in the Hausp^{K444R} mice E14 brain tissue. Staining with anti-Neuropilin-2 antibodies in FIG. 8B showed the diminished Fr structure in 4-week old mice brain tissue.

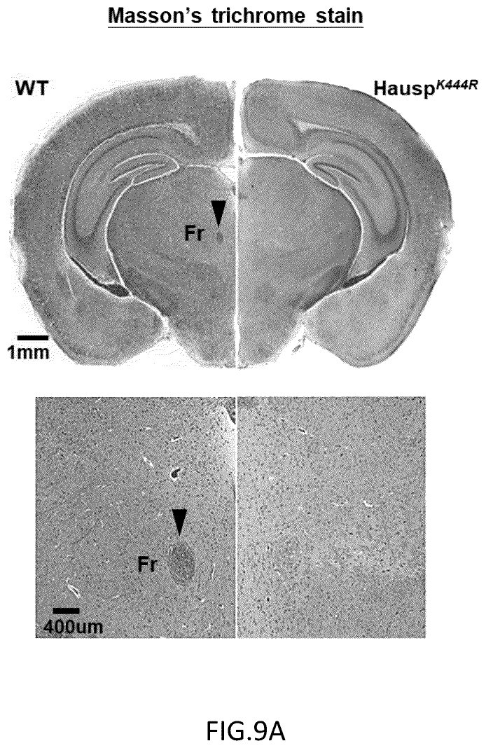

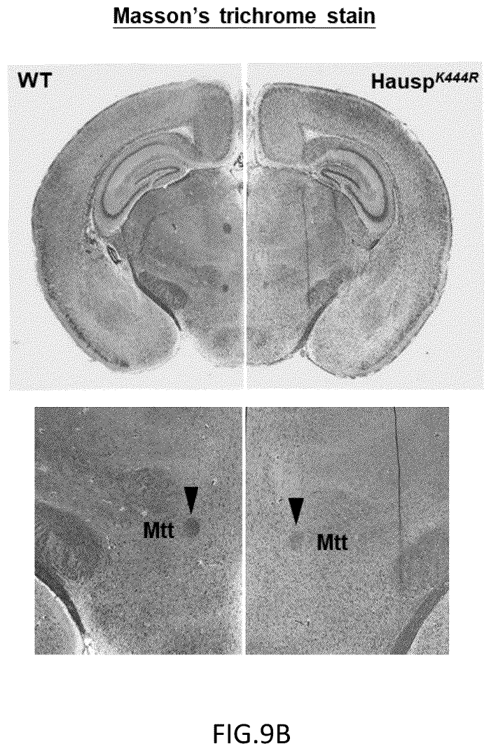

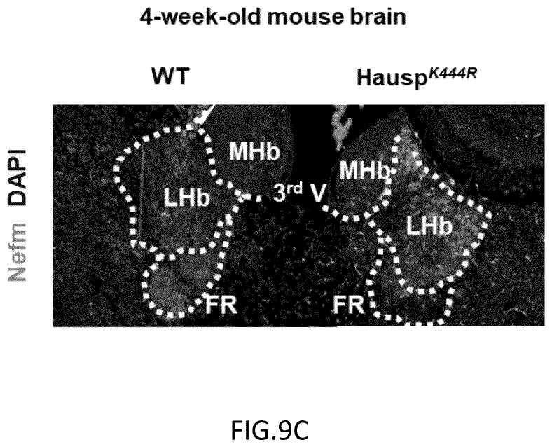

The results of FIG. 9A, FIG. 9B, and FIG. 9C show that the Fr structure was diminished using either Masson's trichrome stain or staining with anti-Nefm antibodies. Masson's trichrome staining of FIG. 9A showed that the structure of Fr was diminished in the Hausp^{K444R} mice brain tissue compared to the wild-type mice brain tissue; whereas the structure of Mtt (Mammillothalamic tract) was not changed between the Hausp^{K444R} mice brain tissue and the wild-type mice brain tissue (serving as a control) in FIG. 9B. Moreover, staining with anti-Nefm (Neurofilament medium chain) antibodies of FIG. 9C show the diminished Fr structure in 4-week old mice brain tissue.

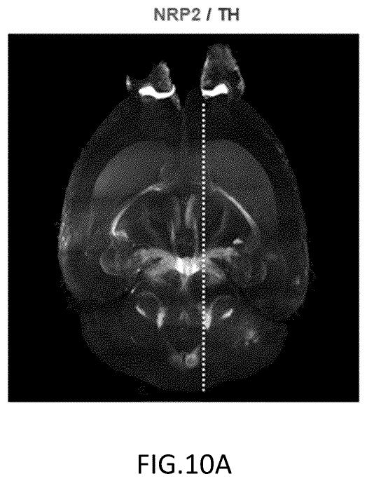

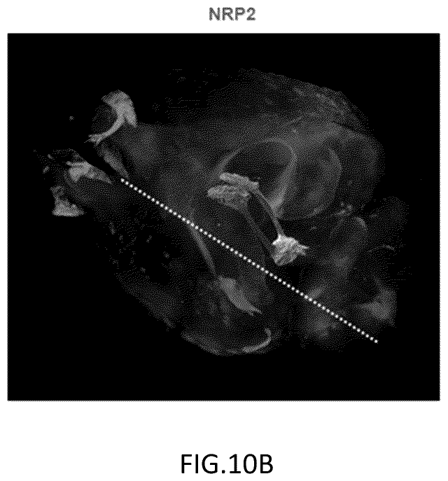

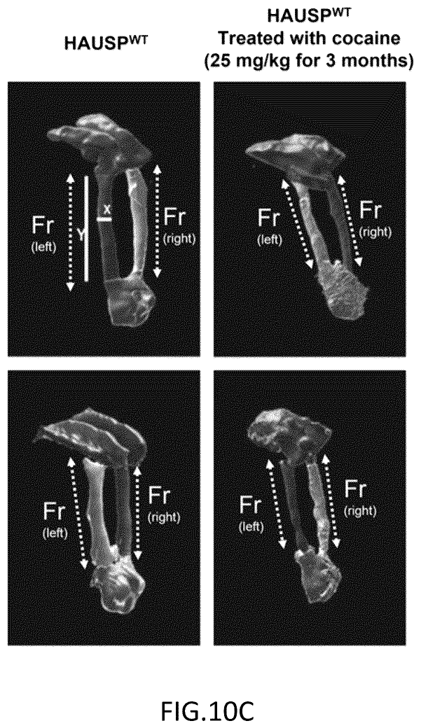

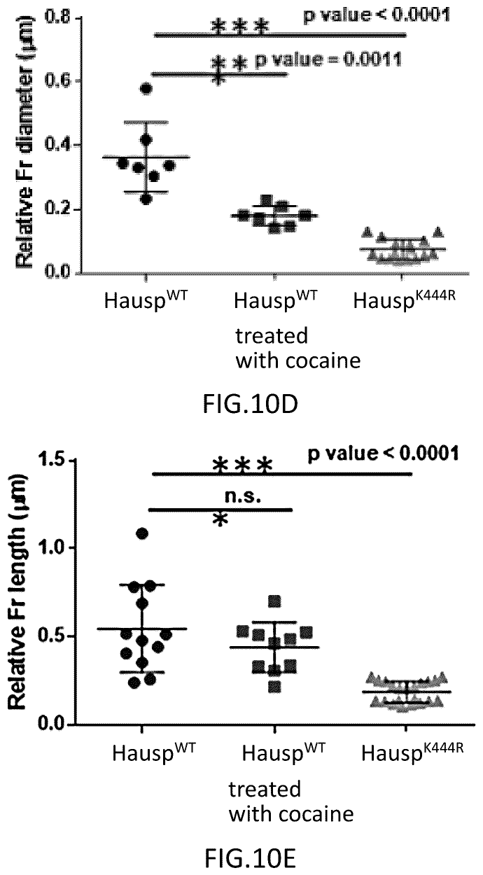

The mouse whole brain tissue staining followed by imaging of FIG. 10A, FIG. 10B and FIG. 10C show the decreased diameter and length of Fr structure in wild-type mice treated with cocaine or the Hausp^{K444R} mice. FIG. 10A and FIG. 10B are examples of wild-type mice whole brain staining with NRP2 (neuropilin-2) with or without TH (tyrosine hydroxylase) followed by imaging. FIG. 10C is an example of the Fr structure in wild-type mice or in the wild-type mice treated with cocaine. The measurement of Fr structure of FIG. 10D and FIG. 10E show a decreased diameter and length of Fr in wild-type mice treated with cocaine or the Hausp^{K444R} mice compared to the control wild-type mice.

Experiment 8: Protein Extraction and Western Blot Assay: SIK2, p35/25, CDK5, p-TH, Co-Immunoprecipitation and GST Pull-Down Assays, and Immunohistochemistry

The protein extraction, western blot assay, co-immunoprecipitation and GST Pull-Down assays are conducted with samples from the mice of Experiment 7 and the experiment methods are similar with Experiment 4. IgG was used as a negative control in Co-immunoprecipitation experiments.

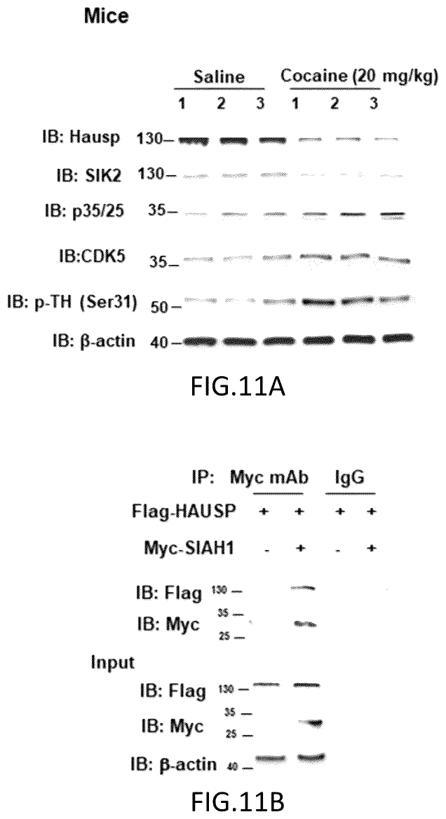

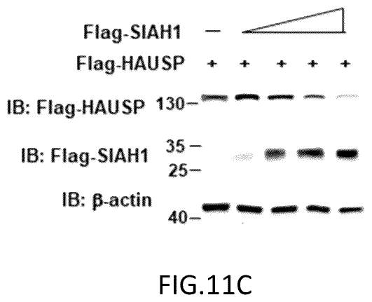

The results of FIG. 11A, FIG. 11B, and FIG. 11C show that the cocaine treatment caused the decrease in Hausp levels in mice brain tissue; and the Siah E3 Ubiquitin Protein Ligase 1 (SIAH1) decreased the HAUSP levels by interacting with HAUSP. The result of FIG. 11A shows that the cocaine treatment decreased the Hausp levels and changed the downstream signaling in mouse brain tissue. Besides, co-immunoprecipitation assays of FIG. 11B show the interaction between HAUSP and SIAH1. Overexpression of SIAH1 decreased the HAUSP levels, as shown in FIG. 11C.

Experiment 9: Protein Extraction and Western Blot Assay: HAUSP, SIK2, p35/25, CDK5 and ΔFosB, and Immunohistochemistry

The human SH-SY5Y cells of 70% confluence were treated with cocaine of 0 to 100 μM combined with/without SIV (100 μg/ml) for 24 hr. Cocaine stock was prepared for 20 mM in ddH₂O and SIV stock (100 mg/ml) was dissolved in DMSO.

The protein extraction and western blot assay were conducted with the protein samples from cell lysate of the aforementioned human SH-SY5Y cells and the experiment methods similar with Experiment 4.

For immunohistochemistry, the pregnant female C57BL/6J mice were treated with SIV three times per week as HAUSP^{K444R}+SIV group, and the mouse embryos were harvested at embryonic day 14 and briefly opened a tiny hole in abdomen followed embedding in 4% paraformaldehyde solution for 24 hours. On the other hand, the wild-type and Hausp^{K444R} mice were treated with saline. After the above treatments, all organs were obtained after mouse heart perfusion by saline, and fixed by 4% paraformaldehyde solution for 24 hr for NrCAM immunohistochemistry (IHC). Fresh tissues were immediately homogenized in T-PER™ by tissue homogenizer (HT mini homogenizer, OPS Diagnostics LLC, USA) for Western blot analysis. Handling of the animals has been approved by the Internal Animal Usage Committee of the Chang Gung Memorial Hospital at Linkou.

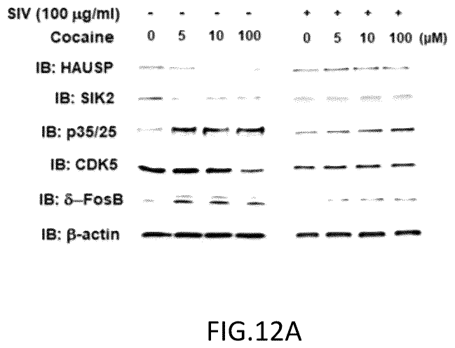

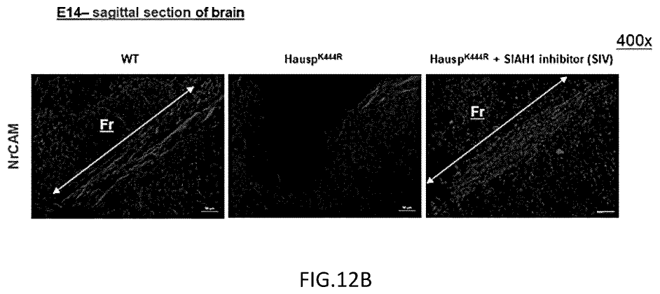

The western blot result of FIG. 12A shows that the SIV restored the HAUSP levels induced by cocaine in SH-SY5Y cells.

The immunohistochemistry result of FIG. 12B shows that SIV treatment rescued the damage of Fr structure and Fr function in the Hausp^{K444R} mice brain compared to the Hausp^{K444R} mice brain without SIV treatment. That is, SIV can be used for neuroregeneration.

Experiment 10: Locomotion Activity and Conditioned Place Preference (CPP) of Cocaine Induced Mice after SIV Treatment

Conditioned place preference (CPP) is a classical conditioning paradigm used in behavioral neuroscience to study the rewarding and aversive properties of drugs or other stimuli. It involves associating a particular environment or location with the experience of a rewarding or aversive stimulus, such as drugs, food, or electric shock. In CPP experiment, mice are first trained to associate one compartment of a box and then, they are placed in the neutral compartment and allowed to freely explore both compartments, and animals' preference for one compartment over the other is measured. After vehicle/SIV (100 mg/kg) treatment for 10 mins, the well-trained mice were administrated with cocaine (25 mg/kg, i.p.) for 30 mins. The content of the vehicle is 0.5% DMSO, 25% PEG-300, 4% Tween-80, and 70.5% saline.

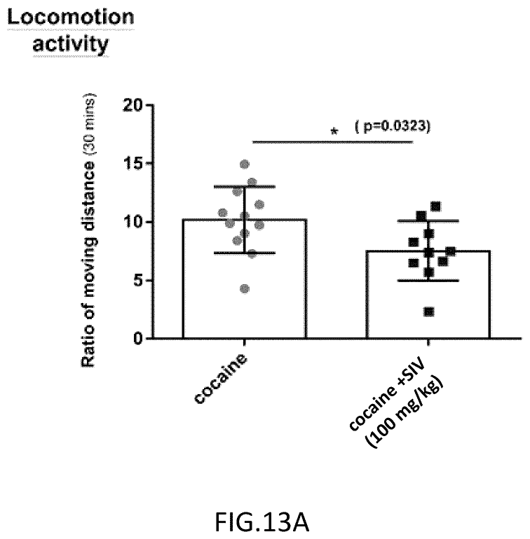

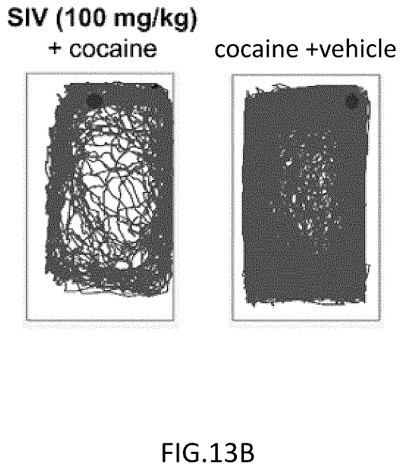

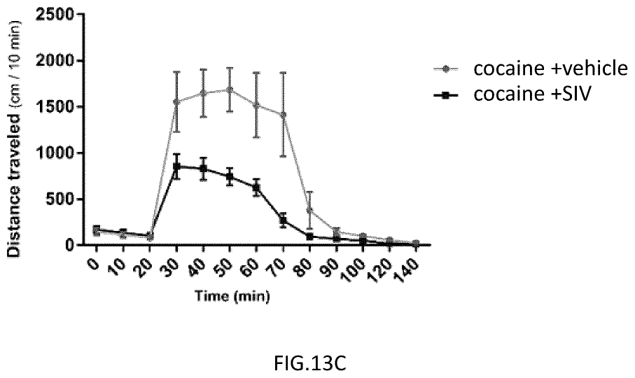

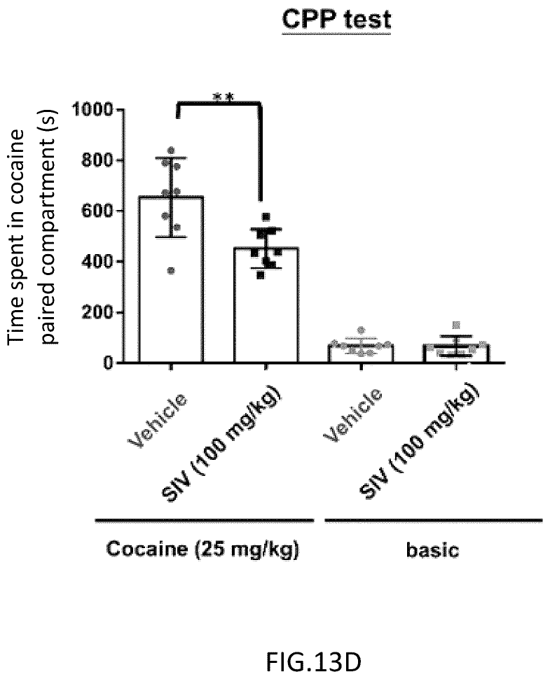

The result of FIG. 13A to FIG. 13D show the influence of SIV on locomotion activity and CPP test induced by cocaine, respectively. After treatment with SIV, increased locomotion activity or increased distance traveled induced by cocaine was decreased (FIGS. 13A to 13C). The CPP test result of FIG. 13D showed that treatment with SIV (100 mg/kg) decreased the time spent in cocaine paired compartments.

### Experiment 11: Lever Press Test

Mice were anesthetized with isoflurane (induction and maintenance at 4%-5%) for surgical implantation of a jugular vein catheter connected to a head-mounted entry port. Briefly, the right jugular vein was exposed, and the catheter tubing was inserted and secured to the vein. The catheter was passed under the skin to an incision on the head, and the port was secured to the skull with dental cement anchored by screws. Mice were given buprenorphine (0.05-0.10 mg/kg, intraperitoneal.) for analgesia and allowed to recover for 7-10 days before beginning self-administration. Catheter patency was maintained by daily infusion of 100 μl heparin (120 U/μl) through the catheter and treated with Cefazolin, 200 mg/kg, subcutaneously, to prevent bacterial infections.

The lever press test was done in extra wide mouse operant chambers with a modified top (Med Associates, VT, USA). The chamber contained a retractable lever, a food pellet receptacle connected to a pellet dispenser, with a stimulus light above the pellet receptacle, and contained a house light. Cocaine was delivered by a syringe pump located outside the chamber, and the drug delivery tubing was connected to the mice through a swivel (Instech, Plymouth Meeting, PA, USA) and the swivel arm connected on top of the chamber to allow free movement of the mice during self-administration. All the sessions were remotely operated and recorded by MED-PC V software (Med Associates, VT, USA). Mice were pre-trained with food through pressing the levers and were food-restricted during the study. After training, mice were provided ad libitum access to water and food.

Self-administration sessions took place once daily during the light-dark cycle for 14 consecutive days. Before starting the study, mice were allowed to acclimate to the housing for approximately 3 days. The availability of cocaine reward was indicated by illumination of the house light and pressing levers. Pressing the active levers resulted in a 4.4-second infusion of cocaine (0.2 mg/kg in a volume of 5 μl, based on weight). Each infusion was paired with a cue (1 s illumination of the stimulus green light) followed by a 10-second time-out signaled by turning off the house light. Pressing the inactive lever was recorded but had no consequences. Each chamber was operated under a 3-hour session, or until mice received 100 infusions (20 mg/kg) of cocaine (to prevent cocaine overdose). No treatment groups were implanted with a catheter and placed in the operant chambers with identical cues but not connected to the syringe pump and received no reward for pressing the active levers. Vehicle or SIV with a dosage of 100 mg/kg was injected intraperitoneally for 10 min prior to cocaine self-administration. The content of the vehicle is 0.5% DMSO, 25% PEG-300, 4% Tween-80, and 70.5% saline. Mice met criteria for acquisition of self-administration when they: (1) self-administered at least 30 infusions in a session; (2) >85% of responses were on the active image; and (3) had no more than 20% variation in number of infusions between sessions. The lever press frequency and number within 3 hours indicate the cocaine addiction symptom.

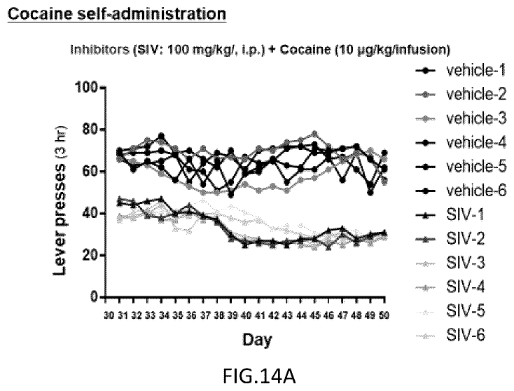

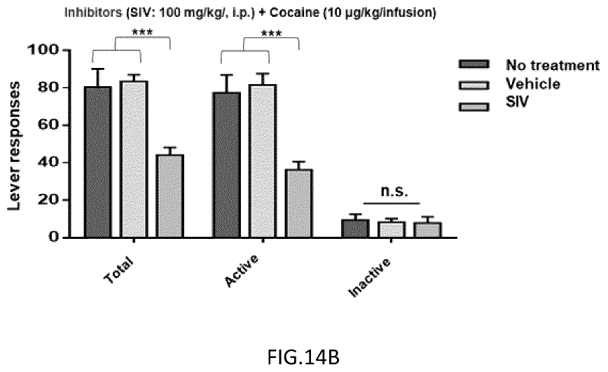

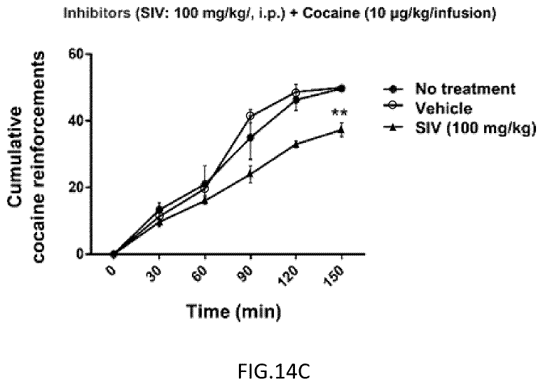

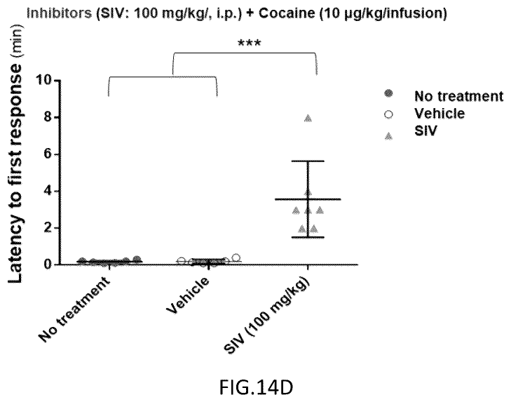

The results of lever press test are shown in FIG. 14A and FIG. 14B, which indicate that the SIV decreased the lever press frequencies induced by cocaine treatment. Besides, SIV (100 mg/kg) treatment decreased the cumulative cocaine reinforcements, which is shown in FIG. 14C. In addition, the result of FIG. 14D shows that the SIV (100 mg/kg) treatment increased the latency to first response in mice treated with cocaine. That is, SIV can prevent and ameliorate cocaine addiction.

### Experiment 12: E3-Ligase Auto Ubiquitination Assay

SIAH1 mediates the K48-linked polyubiquitination of HAUSP. Therefore, for the ubiquitination assay, SIAH1 was incubated in the solution (0.25 mg/1 ml, total 100 μg) containing 20 mM Tris-HCl buffer (pH 8.0), 100 mM NaCl, 1 mM DTT and 40% glycerol and HAUSP (50 μg) was in 50 mM HEPES pH 7.5, 100 mM NaCl, 1 mM TCEP (tris (2-carboxyethyl) phosphine) buffer. Inhibitors (in DMSO, <1%): SIV (50 μg/ml or 100 μg/ml) and vehicle control (abbreviated as VH) were individually co-incubated with SIAH1 and HAUSP in a combined buffer containing 1× ubiquitin activating enzyme solution (E1), 1× ubiquitin, and 1× Ub E3 ligase buffer in 37° C. for 1 hour followed by Western blot analysis using a Ub-K48 antibody (1:1000, Cell signaling, USA.). The E3-ligase auto ubiquitination assay was performed by E3 Ligase Auto-Ubiquitylation Assay Kit (Abcam, ab139469, UK).

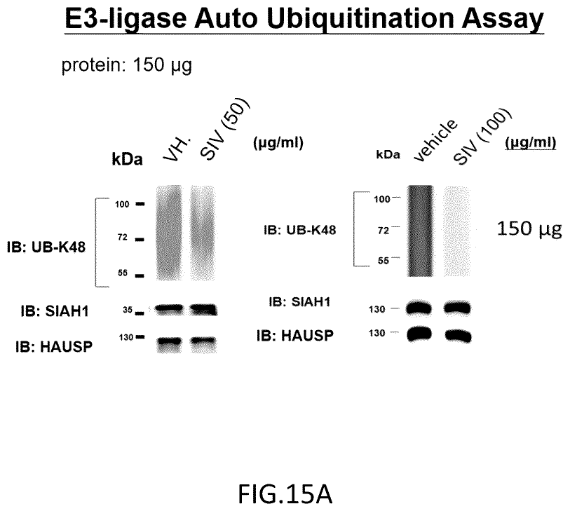

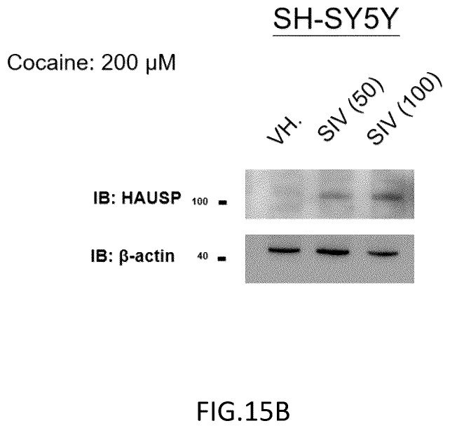

In vitro SIAH1 polyubiquitination assays of FIG. 15A show the ability of SIV (50 μg/ml or 100 μg/ml) to inhibit the K48-linked polyubiquitination of HAUSP mediated by SIAH1. It can be seen that the SIV can inhibit the K-48 linked polyubiquitination of HAUSP, so that the SIV can be used to ameliorate cocaine addiction.

### Experiment 13: Protein Extraction and Western Blot Assay: HAUSP

The human SH-SY5Y cells of 70% confluence were treated with cocaine of 200 μM combined with/without inhibitors (in DMSO, <1%): SIV (50 μg/ml or 100 μg/ml) for 24 hr. Cocaine stock was prepared for 20 mM in ddH₂O.

The protein extraction and western blot assay were conducted as the protein samples from cell lysate of the aforementioned human SH-SY5Y cells and the experiment methods were similar to Experiment 4.

The western blot results of FIG. 15B show that SIV (50 μg/ml or 100 μg/ml) can restore the HAUSP levels decreased by cocaine in SH-SY5Y cells, so that SIV can be used to ameliorate cocaine addiction.

To sum up, SIV can restore HAUSP level in cocaine induced mice, restore the Fr structure and Fr function, decrease the increased locomotion activity or increased distance traveled induced by cocaine, decrease the time spent in cocaine paired compartments, and decrease the increased lever press frequencies induced by cocaine treatment. Therefore, SIV can be used for treating or preventing the cocaine addiction, preventing a relapse of cocaine addiction, neuroregeneration and ameliorating a physical symptom of cocaine addiction.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A method for preventing or treating cocaine addiction, comprising administering an effective amount of a SIAH1 inhibitor to a subject in need thereof.

2. A method for preventing a relapse of cocaine addiction, comprising administering an effective amount of a SIAH1 inhibitor to a subject in need thereof.

3. A method for ameliorating a physical symptom or defect of cocaine addiction, comprising administering an effective amount of a SIAH1 inhibitor to a subject in need thereof.

4. The method as claimed in claim 3, wherein the physical symptom or defect of cocaine addiction is lesion of Fasciculus retroflexus.

5. A method for neuroregeneration, comprising administering an effective amount of a SIAH1 inhibitor to a subject in need thereof.

6. The method as claimed in claim 1, wherein the subject is a human.

7. The method as claimed in claim 2, wherein the subject is a human.

8. The method as claimed in claim 3, wherein the subject is a human.

9. The method as claimed in claim 5, wherein the subject is a human.

10. The method as claimed in claim 1, wherein the effective amount of the SIAH1 inhibitor is 0.01 mg/kg/day to 100 mg/kg/day.

11. The method as claimed in claim 2, wherein the effective amount of the SIAH1 inhibitor is 0.01 mg/kg/day to 100 mg/kg/day.

12. The method as claimed in claim 3, wherein the effective amount of the SIAH1 inhibitor is 0.01 mg/kg/day to 100 mg/kg/day.

13. The method as claimed in claim 5, wherein the effective amount of the SIAH1 inhibitor is 0.01 mg/kg/day to 100 mg/kg/day.

14. The method as claimed in claim 1, wherein the SIAH1 inhibitor is Sivelestat or a pharmaceutically acceptable salt or solvate thereof.

15. The method as claimed in claim 2, wherein the SIAH1 inhibitor is Sivelestat or a pharmaceutically acceptable salt or solvate thereof.

16. The method as claimed in claim 3, wherein the SIAH1 inhibitor is Sivelestat or a pharmaceutically acceptable salt or solvate thereof.

17. The method as claimed in claim 5, wherein the SIAH1 inhibitor is Sivelestat or a pharmaceutically acceptable salt or solvate thereof.
